(19) United States
(12) Patent Application Publication
(10) Pub. No.: US 2016/0213783 A1
(43) Pub. Date: Jul. 28, 2016

(54) PHARMACEUTICAL COMPOSITION AND METHOD OF PREPARATION OF FORMULATIONS FOR THE MANAGEMENT OF DYSPHAGIA

(71) Applicants: HEMANT N. JOSHI, Parsipanny, NJ (US); Harshada N. Sant, Harrison, NJ (US)

(72) Inventors: HEMANT N. JOSHI, Parsipanny, NJ (US); Harshada N. Sant, Harrison, NJ (US)

(21) Appl. No.: 14/607,579

(22) Filed: Jan. 28, 2015

(51) Int. Cl. A61K 47/36; A61K 47/10; A23L 2/52; A61K 47/06

(52) U.S. Cl. CPC A61K 47/36; A61K 47/06; A61K 47/10; A23L 2/52; A23V 2002/00

(57) ABSTRACT

The present invention proposes a formulation and the process to prepare thick water to be administered to patients afflicted by dysphagia. It particularly comprises of a polymer that helps the formulation to stay thick when administered and thins out as it passes to the stomach. The formulation does not regain viscosity when it enters duodenum and small intestine regions. This formulation for the management of dysphagia can be prepared at any consistency/viscosity required. Some of the advantages proposed are a decrease in feeling of satiety, ease of administration and no effect on the bioavailability of co-administered medications.

The present invention relates to the field of managing dysphagia—a difficulty in swallowing liquids. It presents composition and the method of preparation of formulation which can be used to manage dysphagia. Thickened water is normally administered to patients suffering from dysphagia.

### BACKGROUND OF THE INVENTION

Dysphagia is a term commonly used for people who have difficulty in swallowing liquids as well as solids to some extent. The esophagus squeezes or contracts rhythmically to let the food or liquids move from mouth to the stomach. Dysphagia indicates the inability of esophagus to squeeze or contract due to several reasons which include stroke, brain or spinal cord injury, muscular dystrophy, Parkinson's disease, esophageal spasm, scleroderma. The blockage of esophagus, for example, due to cancerous growth in esophagus, may also cause the food and liquids to have difficulty in passing through the esophagus. Esophageal spasm may be a result of Gastro Esophageal Reflux Disease (GERD) or Achalasis. GERD is a problem with the nervous system causing improper functioning of the muscles of the esophagus and the lower esophageal sphincter.

General signs of dysphagia may include: A. coughing during or right after eating or drinking, B. wet or gurgly sounding voice during or after eating or drinking, C. extra effort or time needed to chew or swallow, D. food or liquid leaking from the mouth or getting stuck in the mouth, E. recurring pneumonia or chest congestion after eating, F. weight loss or dehydration from not being able to eat enough.

Because of dysphagia, patients may have: A. poor nutrition or dehydration, B. risk of aspiration (food or liquid entering the airway), which can lead to pneumonia and chronic lung disease, C. less enjoyment of eating or drinking, D. embarrassment or isolation in social situations involving eating.

Dysphagia is classified into three major types—1. Oropharyngeal dysphagia, 2. Esophageal dysphagia and 3. Functional dysphagia. Oropharyngeal dysphagia arises from abnormalities of muscles, nerves or structures of the oral cavity, pharynx, and upper esophageal sphincter. Esophageal dysphagia arises from the body of the esophagus, lower esophageal sphincter, or cardia of the stomach (area immediately surrounding the opening from the esophagus into the stomach), usually due to mechanical causes or motility problems. Functional dysphagia is the sensation of solid and/or liquid foods sticking, lodging, or passing abnormally through the esophagus. In some patients, no organic cause for dysphagia is found.

People suffering from dysphagia have no muscle control and are unable to co-ordinate the closing of wind pipe while swallowing the food or liquids. The entry of food or liquids in lungs might give rise to growth of bacteria, which may lead to aspirational pneumonia or asphyxiation. Thickening the liquids consumed by these people, which provides better bolus control and oral stimulation, is a common approach towards solving the problem.

Modified barium swallow (video fluoroscopic swallow study), Fiber-optic endoscopic swallowing evaluation, imaging tests like computerized tomography (CT) or magnetic resonance imaging (MRI) scans, endoscopy, esophageal manometry, and biopsy are few tests to be performed for correct diagnosis of dysphagia in patients and to decide the course of action.

“Disease Management” is defined as “a system of coordinated healthcare interventions and communications for populations with conditions in which patient self-care efforts are significant”. The goal is to improve the quality of life of patients by preventing or minimizing the effects of disease. In the case of dysphagia, thickened water does not cure the disease, but it allows patients to drink water, which is crucial for survival. Also, the thickened water does not go to the wind pipe and enter esophagus. It thereby prevents further complications such as pulmonary infections. Therefore, the term “management of dysphagia” is used in the current patent application.

Thickened beverage compositions have been introduced for people suffering from dysphagia. Commonly used thickeners are xanthan gum or starch. Currently products such as Simply Thick® and Resource® ThickenUp® are sold in market. Simply Thick® is a gel-based thickener, which consists of water, xanthan gum, citric acid and potassium sorbate. It is to be added to water to obtain desired consistency like nectar thick (1 strokes/4 oz), honey thick (2 strokes/4 oz), or pudding thick (4 strokes/4 oz). Resource® ThickenUp® is a powder-based thickener, which has to be reconstituted with water to desired consistency. It contains modified food starch (Corn). Resource® ThickenUp® Clear consists of maltodextrin, xanthan gum, and potassium chloride, which yields clearer liquid than the previous one. Thick & Easy® Clear Food Thickener is a powder-based thickener which has maltodextrin, xanthan gum, carrageenan and erythritol. Thick & Easy® Food Thickener is another powder-based thickener, which contains modified food starch and maltodextrin. The product is also available as a pre-thickened beverage. Thick It® contains maltodextrin and modified corn starch, which can be reconstituted easily without lumps. Thick It-2® is more concentrated than Thick It® so that the amount used for reconstitution is less. Thick & Clear® is a starch-free thickener, which has standardized cellulose gum and dextrin. There are certain pureed food thickeners too but they are not used as frequently as the gel and powder-based thickeners to manage dysphagia.

Though starch thickens the liquids, it has some problems, which limits its use as a thickener. Starch-based thickeners are found to impart a starch flavor and a grainy texture for nectar- and honey-thick consistencies. Also starch being a carbohydrate, adds caloric value to the diet, which may not be recommended for people on low calorie diet. Eating too much starch is not advisable for people with diabetes. Starch has gluten, which makes it unsuitable for patients of celiac's disease. Also digestible carbohydrates are limited in ketogenic diets for controlling epileptic seizures, so the people included in the category cannot have starch-thickened water. Palatability of starch thickened beverages is not good to some patients.

Xanthan gum is composed of pentasaccharide repeat units, comprising glucose, mannose, and glucuronic acid in the molar ratio 2.0:2.0:1.0. Xanthan gum-based thickeners do not produce grainy textures, but produce a higher ‘slickness’ than starch-based thickeners. Xanthan gum containing products work well but can cause a feeling of satiety and the patient might not feel hungry after consuming the thickened water.

Conventional formulations to administer water or food to people suffering for dysphagia provide a feeling of satiety or full stomach. It might lead to less intake of food consisting of essential nutrients by the patient. In most cases the patients are old and hence they might suffer from weakness and malnutrition. Secondly, the bioavailability of the medications administered with the conventionally thickened beverages may get affected as the high viscosity would hinder the release of medication. Especially for tablet dosage form, the first step in absorption of actives is disintegration, which needs absorption of water in the tablet. Water in thickened water formulation in a molecularly bound form and is not freely accessible for tablet disintegration. Most of the old patients take many medicines and the change in bioavailability of drugs is not desirable. Thick liquids can also slow down the motility of gastro-intestinal tract. Hence there is a need of a product, which will help dysphagia patients to drink water but should not produce above-mentioned side effects.

US patent application #20040258823 claimed a method for preparing adapted food compositions for facilitating the swallowing of food for people suffering from dysphagia. It stated a method of modifying the food substance with the use of binding, gelling or thickening agents. It specified a dysphagia diet, which may include minced diet, pureed diet or minced-pureed diet. The said dysphagia diet also included thickened liquids, which could be of honey-, nectar- or pudding-consistency depending on the type a degree of dysphagia the person suffering from. U.S. Pat. No. 8,445,044 was similar and it provided a food and water thickener and a method for preparing the food and water thickener. The food thickener comprised pretreated mineral water, xanthan, food preservative, a first antimicrobial and chelating agent, and a sequestering agent, wherein the food thickener had a density of between 750 and 1250 grams per liter.

US patent application #20060051296 (issued U.S. Pat. No. 8,481,000) invented thickened beverage compositions for the management of dysphagia. They proposed the use of xanthan gum, guar gum and some cellulosic polymers. US patent application #20090074940 proposed thickened nutritional products for consumption by dysphagia patients. The compositions comprised starch, xanthan gum and/or methyl cellulose and galactomannan and/or glucomannan. US patent application #20110135799 was a continuation of work from the US patent application #20060051296. It described the production of thickened beverages by a dispensing machine that is also capable of dispensing non-thickened beverages. US patent application #2011/0217442 proposed a composition suitable for dysphagia patients. It used modified xanthan gum. The modified xanthan gum comprised of a non-pyruvylated xanthan gum, a reduced pyruvylated xanthan gum or a combination thereof. US patent application #2012/0258195 is a continuation patent of US patent application 20090074940 proposed compositions comprising: (a) 40-70 wt. % starch; (b) 1-5 wt. % xanthan gum, methylcellulose, or both; (c) 4-20 wt. % tara gum; and (d) 15-55 wt % maltodextrin. US patent application #20140024581 pertains to a liquid enteral nutritional composition with an energy density between 1.0 and 4.0 kcal/ml, a viscosity between 150 and 1800 mPas measured at a shear rate of 50/second at 20 degree C., comprising digestible carbohydrates and fat, wherein the composition further comprises at least one of (a1)) between 8-20 g protein per 100 ml of the composition, where micellar casein comprises at least 50 wt % of the total protein content of the composition, or (a2) between 16-45% protein, where micellar casein comprises at least 50% of the protein caloric content; and (b) anionic fibers capable of sequestering of calcium, and (c) carrageenan between 0.015 and 0.25 g per 100 ml of the composition, and its use for preventing/treating dysphagia, and/or treating/preventing malnourishment or undernourishment associated with dysphagia. U.S. Pat. No. 8,623,323 claimed thickened beverage compositions for management of dysphagia. The ready-to-consume product or the thickener concentrate included the use of soluble food fiber thickener. The patent specifically described usage of preferred food thickener—xanthan gum. A concentrate of this thickener was prepared, which could be used as beverage as well as a food thickener fulfilling the demand of water. This patent did not mention about overcoming the feeling of satiety.

U.S. Pat. No. 8,696,568 stated methods, system and kit for diagnosing and treating dysphagia. The method included screening the patient for dysphagia symptoms, diagnosing and categorizing the dysphagia if the patient exceeded the threshold symptoms and providing a physical therapy regime depending on the severity of dysphagia. It further stated that subjective measurement like stirring or oral feel was not enough, and an objective measurement should be conducted to ensure the correct viscosity measurement.

A review article, ‘Thickening agents used for dysphagia management: effect on bioavailability of water, medication and feelings of satiety’, explains the differentiation of the consistency of water based on the viscosity of the liquid (Cichero Nutrition Journal 12: 54-60, 2013). Table 1 lists details on the products with various viscosities.

TABLE 1
Levels of Fluid Thickness commonly used in Dysphagia Treatment

| | | Moderate | |
|---|---|---|---|
| Parameters | Least Thick | Thickness | Most Thick |
| Labels used | Nectar Thick | Honey Thick | Spoon Thick |
| Level | Level 150 | Level 400 | Level 900 |
| | Mildly thick | Moderately thick | Extremely thick |
| Viscosity | 51-350 | 350-1750 | >1750 |
| range (mPa · s) | | | |

Note:
1 centipoise (CPS) = 1 mPa · S = 1 Centistokes (cSt)

For the purpose of the current patent application, the “Thickened water” is defined as the water whose viscosity is increased more than 10 cps or 10 cSt by the addition of a suitable thickening agent. The bioavailability of water was previously reported to be unaffected by the thickness. But the thickened water reported to affect the bioavailability of medicines administered concomitantly. Thickened water was observed to impede dosage form disintegration and drug dissolution. Normally, patients tend to consume less amounts of thickened liquids compared to plain water. Therefore, the viscosity of the thickened fluid should be optimal. Thickened water tends to keep high air loading, which in turn causes satiety. The incorporation of air in such product has to be kept to a minimal level. The sensation of feeling full has other clinical implications. The entrapped air can also uneasiness in the gastro-intestinal tract.

The key objective of the present invention is to prepare a formulation, which is a thickened liquid useful in the treatment of dysphagia but should overcome the disadvantage of satiety once it reaches stomach. This in turn, would overcome other issues such as reduced bioavailability of medicines.

### SUMMARY OF THE INVENTION

The current invention states the use of thickened water for the management of dysphagia. The proposed product is expected to be devoid of the side-effects normally observed with the marketed dysphagia products. The proposed product is a clear liquid, it has an appropriate viscosity necessary for the thickened liquids, acceptable palatability and satisfactory taste in the mouth. The stated thickened water can be of any consistency such as nectar thick (150 cSt), honey thick (400 cSt) or spoon thick (900 cSt). The novelty about the proposed thickened water is that the viscosity of the water while administration is high and after administration, the viscosity of the water decreases in the stomach. This way, the person does not get a feeling of satiety (normally observed with the commercial products) after drinking the proposed thickened water. This will not also affect the motility of GI tract and will not affect the bioavailability of medicines administered concomitantly to the patient. Hence the product serves the purpose of allowing the thickened water to enter only in the esophagus and not in the wind pipe and avoids the feeling of fullness caused in the stomach.

In an embodiment, the proposed product is a ready-to-use liquid comprising a suitable thickening agent, water and neutralized with an alkali.

In an embodiment, the proposed product is a ready-to-use liquid comprising a suitable thickening agent, a suitable preservative, and water. The product is neutralized with an alkali.

In other embodiments, the pharmaceutical composition is in a powder form. This powder can mixed with water provided with the product to produce thickened water.

It yet another embodiment, the proposed product is a powder mixture. The powder composition is mixed with a suitable concentration of an alkali in water to produce thickened water. The powder formulations can be produced using a granulating liquid. A “granulating liquid” is understood herein as a binding agent added to bind the fine particles into granules. The granulating liquids may include, but are not limited to water, ethyl alcohol or hydro-alcoholic mixtures.

In certain embodiments, the powder composition may also contain a “diluent” or a “granulating agent”. A “diluent” or a “granulating agent” is a substance that gives volume to the powder composition for dysphagia management maintaining its palatability. Such granulating agents include, but are not limited to, lactose, sucrose, dextrose, mannitol, sorbitol, cellulose and its derivatives (e.g., microcrystalline cellulose, carboxymethyl cellulose calcium, sodium carboxymethyl cellulose, hydroxypropyl methyl cellulose etc.), calcium sulfate, dibasic calcium phosphate, polyvinyl pyrrolidone and a mixture of any of the above thereof.

In certain embodiments, the powder composition may also contain a neutralizing agent/alkali. A “neutralizing agent/alkali” herein means certain embodiments, which are substances that are added to decrease the acidity or increase the pH of the solution. A type of “neutralizing agent/alkali” would include, but are not limited to, potassium hydroxide, sodium hydroxide, calcium hydroxide, calcium carbonate, magnesium oxide, ammonia, triethanolamine and other low molecular weight amines and alkanolamines.

In yet another embodiment, a preservative or a mixture of preservatives is added to the thickened water formulation. A “Preservative” refers to a substance that is added to prevent microbial growth. The preservative include but are not limited to methyl paraben, propyl paraben, thiomersal, chlorocresol, benzalkonium chloride, sodium benzoate, benzoic acid, sodium nitrate, sodium nitrite, quaternary ammonium chloride, potassium nitrate, potassium sorbate, natamycin, heptyl paraben, sorbic acid, acetic acid, propionic acid, sodium bisulfite, and mixtures thereof.

In other embodiment of the invention, suitable coloring and flavoring agents can be used to make proposed formulations palatable and acceptable by patients.

In yet another embodiment of the invention, suitable sweeteners can be used to make the proposed formulation palatable and acceptable by patients. The sweeteners can be natural or artificial, commonly known to people working in the field. The sweetener include but are not limited to acesulfame potassium, aspartame, cyclamate, etythritol, glycyrrhizin, isomalt, lactitol, maltitol, mannitol, neotame, saccharin, sucralose, tagatose, xylitol, sorbitol, and mixtures thereof.

### BRIEF DESCRIPTION OF THE DRAWINGS

FIG. 1 shows the effect of pH on the viscosity of the formulation. The product showed the viscosity of 1957 cSt at pH 6.92. The addition of 0.5 N hydrochloric acid decreased the pH and the viscosity dropped significantly (40 cSt at pH 6.18). The pH was further decreased to 5.02 and the observed viscosity was observed to be 4.17 cSt. The pH was reversed back to 7.06 by the addition of 1 N sodium hydroxide, but the viscosity did not increase back to the initial value, but remained at 17.4 cSt.

### DEFINITION OF TERMS USED

Unless defined otherwise, all technical and scientific terms used herein have the same meanings as commonly understood by one of ordinary skill in the art.

As used herein and in the claims, the singular forms “a”, “an”, and “the” include plural reference unless the context clearly dictates otherwise. For example, reference to “an excipient” is a reference to one or more excipients and equivalents thereof known to those skilled in the art.

The term “about” is used to indicate that a value includes the standard level of error for the substance or method being employed to determine the value. The terms “comprise,” “have” and “include” are open-ended linking verbs. Any forms or tenses of one or more of these verbs, such as “comprises,” “comprising,” “has,” “having,” “includes” and “including,” are also open-ended.

The terms “treat,” “treated,” “treatment,” or “treating” used herein refers to both therapeutic, prophylactic or preventative measure to prevent or slow (or lessen) an undesired physiological condition, disorder or disease, or to obtain beneficial or desired clinical results.

“Dysphagia” refers to condition, which can cause disruption in the swallowing process interfering with patient's ability to eat or drink. It can result in aspiration pneumonia, malnutrition, dehydration, weight-loss, and airway obstruction.

“Excipients” are compounds used in the dosage form along with the active ingredient. The drug has to stable in the dosage form along with the excipients throughout the shelf-life of the pharmaceutical dosage form. In this patent application, a thickening agent is the substance providing therapeutic effect to the formulation. Thus, it can be termed as the “active” ingredient and all other ingredients would be considered as “excipients”.

“Pharmaceutically Acceptable Materials” refers to those compounds or materials which are suitable for use in contact with tissue or organs of humans and animals without excessive toxicity, irritation, allergic response or any other problems. Only pharmaceutically acceptable materials were used in the formulations prepared in this patent application.

“Chemical stability” with respect to the therapeutic agent means that an acceptable percentage of degradation products are produced by chemical pathways such as hydrolysis, thermal degradation or oxidation. In this formulation, loss of stability will result in the loss of thickening power of the “active” substance used.

“Physical stability” with respect to the therapeutic agent means that an acceptable percentage of aggregates, loss of smell or generation of foul smell, loss of original color or discoloration, crystals, visible mold/fungus is formed. In liquid formulations, no mold/fungus growth is desired and that is why the presence of an antibacterial agent is essential in these types of formulations.

The term pH expresses the acidity or alkalinity of a solution on a logarithmic scale. The pH value of 7 is assigned to a neutral solution. The pH of the solution, in general, has a significant impact on the chemical degradation of the components of the formulation. In these formulations, the pH has a significant impact on the thickening property.

“Polymer” is a substance that has a molecular structure consisting chiefly or entirely of a large number of similar units bonded together.

“Acid-functional Polymer” or “anionic polymer” is referred to as a polymer characterized by a surface active negatively charged ions. Various anionic polymers can be used in this product, one of them being carbomer.

“Carbomer” relates to synthetic high molecular weight polymers of acrylic acid. There are different grades of Carbomer available—Carbomer homopolymer type A, Carbomer homopolymer type B, Carbomer homopolymer type C, Carbomer copolymer type A, Carbomer copolymer type B, Carbomer 934, Carbomer 934P, Carbomer 940, Carbomer 941, Carbomer 1342, Carbomer 71 G etc.

“Viscosity” is defined as measure of resistance of a liquid to deformation by shear or tensile stress. The instrument used to measure viscosity is called as a “viscometer”. In the current patent application, Cannon viscometer was used to measure viscosity values. The viscometer provides “kinematic” viscosity values.

“Satiety” is referred to as the satisfied feeling of fullness in the stomach after eating. Early satiety is feeling full inspite of eating or drinking very less amount of food or water, respectively.

“Deionized water (DI)” or “demineralized water” is the water, which is rendered free of ions by using an ion exchange process. At the same time, the deionized water has hydrogen and hydroxyl ions present which make water. DI water can be produced by various techniques. For the purpose of this invention, DI water should have the resistivity at 25° C. of more than 10 MO-cm.

“Thickener mixture” or “powder mixture” is the mixture of powders, which when reconstituted with alkaline deionized water yields the desired thickened water product.

“Alkaline deionized water” is defined as the deionized water whose pH is increased to a basic region (pH 7 to pH 14) with an addition of a suitable alkali.

“Therapeutic Water” is water for a specific therapeutic purpose. The term “therapeutic” relates to the treatment of disease or the action of remedial agents. In this proposal, the thickened water can be termed as the therapeutic water.

“Ready-to-use” phrase is self-explanatory. It is a product which can used “as is” without any further change.

“Stomach”, “Duodenum” and “Small intestine” are the parts of the human gastrointestinal tract. When we eat or drink, the contents pass through the esophagus to stomach, to duodenum, to small intestine and then to large intestine. The pH of contents of stomach is acidic (1.5 to 3.5), but may increase or decrease when we eat depending upon the kind of food we consume. The duodenal pH ranges from about 4.5 to about 6.5 and the pH of small intestine ranges from about 6 to about 7.4. There are many factors affecting the pH values in different parts of the gastro-intestinal tract.

“Bioavailability” of a medication refers to the rate and extent of the drug absorption into the systemic circulation.

### DETAILED DESCRIPTION

The current patent application describes the composition of products used in the management of dysphagia. Patients suffering from dysphagia have difficult in swallowing water, but can swallow thickened water. The viscosity of thickened water is higher than that for the plain water. The kinematic viscosity of plain water is 1 cSt at 20° C. The viscosity of a substance or product can be determined by various techniques. Cannon Fenske Routine Viscometer is used to measure the kinematic viscosity of transparent Newtonian liquids while Cannon fenske opaque viscometer is used for measuring kinematic viscosity of dark Newtonian fluids. There are different Cannon viscometers used depending on the viscosity of the liquid to be measured. Table 2 lists the viscosity ranges for different Cannon viscometers.

TABLE 2
List of Cannon viscometers and their respective viscosity ranges.

| Viscometer | Range of viscosity (cSt) |
|---|---|
| 75 | 1.6 to 8 |
| 100 | 3 to 15 |
| 150 | 7 to 35 |
| 200 | 20 to 100 |
| 300 | 50 to 250 |
| 350 | 100 to 500 |
| 400 | 240 to 1200 |
| 450 | 500 to 2500 |

There are two main components in the thickened water—water and a polymer or mixture of polymers. The product can be marketed as a liquid Ready-to-Use formulation or as a powder/granules mixture (thickener mixture), which can be mixed with water just prior to administration. The composition can also contain excipients such as antimicrobial agent, coloring agent, flavoring agent, sweetener and a suitable diluent. The percent of polymer necessary in the thickened water depends upon the polymer used and the consistency needed by the physician for the treatment. Use of antimicrobial agent, particularly in the Ready-to-Use Thickened Water is necessary, water being a rich source for growth of bacteria. Addition coloring and flavoring agents and sweeteners can increase the patient acceptability of the Thickened water.

Anionic polymers have been used in this composition. Following is the list of anionic polymers and their salts which can be used in the proposed formulations: A. Carboxylic acid polymers-poly(acrylic acid), poly(acrylic acid) ammonium salt, poly(acrylic acid) sodium salt, poly(butadiene/maleic acid), poly(n-butyl acrylate/acrylic acid), poly(ethyl acrylate/acrylic acid), ethyl acrylate/methacrylic acid copolymer, poly(ethylene/acrylic acid[92:8]), polyethylene/maleic anhydride), poly(maleic acid), poly(methacrylic acid), poly(methacrylic acid) ammonium salt, poly(methacrylic acid) sodium salt, acrylic acid/isooctyl acrylate copolymer, polycarbophil calcium, carbomer homopolymer type A, carbomer homopolymer type B, carboxypolymethylene carbomer, methacrylic copolymer type A, methacrylic acid copolymer type B, methacrylic copolymer type C, vinyl acetate/crotonic acid copolymer. B. Phosphoric acid polymers—poly (vinyl phosphoric acid) sodium salt, and C. Sulfonic acid polymers—poly (styrenesulfonic acid), poly (styrenesulfonic acid) sodium salt and alginic acid/sodium alginate.

In certain embodiments, a powder mixture for the thickened water can be prepared as granules using certain polymer and filler/sweetener, granulating it with a granulating liquid. Polymers can be used alone or in combination without implying limitation to the ones included in the list.

Liquid Ready-to-Use Compositions

As the name suggests, the liquid composition is ready-to-use by the patient. The formulations in general are prepared by dissolving the polymer in water and then adjusting pH to a desired value. In some examples, polymer can be added to a mixture of alkali and water (Table 3).

### Example 1

### Carbomer Solution

TABLE 3
Ready-to-Use Thickened Water with Carbomer

| Ingredient | Percent |
|---|---|
| Carbomer | 0.12 |
| Neutralizing agent/alkali | 0.8 |
| Deionized Water | 99.08 |

Table 3 lists the composition of ready-to-use thickened water. Carbomer was added to a premixed alkali-water mixture and allowed to sit for sufficient time till the carbomer got wetted and dispersed in the water forming a clear gel. The gel was thicker than water and showed good flowability. The viscosity was observed to be 990 cSt.

In the following example (Table 4), Alginic acid was used instead of Carbomer. Sufficient quantity of neutralizing agent or alkali was added to obtain a desired pH of the formulation

### Example 2

### Alginic Acid Solution

TABLE 4
Liquid Ready-to-Use Thickened Water with Alginic acid

| Ingredient | Percent |
|---|---|
| Alginic acid | 0.9 |
| Neutralizing agent/alkali | q.s |
| Water | q.s |

The phrase “q.s.” stands for “quantity sufficient”. In terms of alkali, sufficient amount is added to attain a desired pH. A typical pH range is 6 to 8. Sufficient water is added to make up the volume to 100%.

The same polymer can be available in various grades, which can produce different viscosity values. The same grade of polymer from different suppliers can produce different viscosities. There is also batch-to-batch variation within a particular grade of polymer from the same supplier. Thus, caution must be exercised to prepare this product.

Powder Mixtures

Sometimes it is beneficial to prepare thickened water on-the-spot just prior to administration. In the powder form, there may not be a need to add an anti-microbial agent to the formulation. However, one has to consume the formulation within limited time. Also, there is a need of a qualified person to prepare the formulation. Table 5 lists the composition of powder mixture formulation of thickened water.

### Example 3

### Carbomer Powder Mixture

TABLE 5
Composition of a Powder Mixture with Carbomer

| | |
|---|---|
| Ingredient | Percent |

Preparation of Powder Mixture

| | |
|---|---|
| Carbomer | 0.12% |
| Sorbitol | 1% |
| Ethyl Alcohol | Quantity sufficient for |
| | granulation |

Preparation of Thickened Water

| | |
|---|---|
| Powder Mixture | 1.12 |
| Water with Alkali | q.s. to 100% |

This method involved faster dissolution of the carbomer in water with the aid of a granulation process. The carbomer/sorbitol mixture was granulated with ethyl alcohol and dried. Completely dried granules were added to the alkali-water mixture. The gel was formed within minutes of addition of granules. The viscosity of resultant formulation was 2499 cSt. A formulation was also prepared by physically mixing polymer with sorbitol (with no granulation) as a control. The gelling time was significantly longer compared to the time required for the granules of carbomer with sorbitol.

In certain embodiments, effect of concentration of carbomer in water on the viscosity was examined. The concentration of polymer was between 0.04 to 0.12%. The pH of the formulation was adjusted to about 7.0 using an alkali. The viscosity value increased with an increase in the concentration of the polymer (Table 6). The pH of the formulation was decreased with an addition of hydrochloric acid and the viscosity values were measured. The viscosities decreased as the pH of the formulation was decreased. For example, the viscosity of 0.12% Carbomer in water was 852.6 cSt at pH 7.10. When the pH was lowered to 6.05, the viscosity decreased dramatically to 12.3 cSt. Similar effect was observed for other concentrations too. The pH of contents in the stomach is in the range 2 to 5. It means, when the thickened water would reach stomach, it would be exposed to an acidic pH. Based on the data in Table 6, it is clear that the viscosity of thickened water will decrease as soon as it reaches the stomach. The pH of these solutions was increased with the addition of alkali. However, the viscosity did not increase back to the original value. This is very important as the contents of stomach are delivered to duodenum and then to small intestine where the pH increases to a neutral range. There is a possibility of solution becoming viscous with an increase in the pH, but that was not observed.

TABLE 6
Effect of pH on the viscosity of Thickened Water with Carbomer

| | | | | | |
|---|---|---|---|---|---|
| | Viscosity# | | Viscosity# | | Viscosity# |
| pH | (cst) | pH | (cst) | pH | (cst) |

| | | |
|---|---|---|
| 0.085% | 0.1% | 0.12% |

| | | | | | |
|---|---|---|---|---|---|
| 7.00 | 81.85 | 7.00 | 299 | 7.10 | 852.6 |
| 6.07 | 7.68 | 5.33 | 4.48 | 6.05 | 12.37 |
| 3.7 | 1.09 | 4.25 | 1.63 | 3.34 | 0.94 |

#Viscosity values were measured using cannon fenske viscometer 100-400.

In certain embodiments, the effect of temperature on the viscosity of thickened water was studied. The conducted study showed an increase in viscosity at lower temperatures, but the change was not significant (Table 7).

TABLE 7
Effect of temperature on the viscosity of Thickened Water

| Temperature | Viscosities |
|---|---|
| 5° C. | ~300 cst |
| Room Temperature | ~220 cst |
| 40° C. | ~200 cst |

In a powder mixture, the polymer was mixed with a diluent and then granulated. The diluent or the granulating agent may include, lactose, sucrose, dextrose, mannitol, sorbitol, cellulose and its derivatives (e.g. microcrystalline cellulose, carboxymethyl cellulose calcium, sodium carboxymethyl cellulose, hydroxypropyl methyl cellulose), calcium sulfate, and dibasic calcium phosphate, polyvinyl pyrrolidone, and mixtures thereof. The powder mixture is granulated using granulating liquid such water, anhydrous alcohol or alcohol water mixture, but not limited to only these solvents. The prepared granules can be added to alkali water where the alkali/neutralizing substance can be, but not limited to potassium hydroxide, sodium hydroxide, calcium hydroxide, calcium carbonate, magnesium oxide, and ammonia/ammonium hydroxide.

In an embodiment, the packaging may include separately packed alkali water and the granules, which are to be reconstituted when required. In this case, the resultant pH of the thickened water is in the range 5 to 8. Another aspect may include packaging of Ready-to-Use thickened liquids in an appropriate bottle labelled accurately.

In certain cases, the bottle may have a suitable device enabling the patient to drink the water properly. In an aspect, when the viscosity of liquid is too high for certain severe cases, the water can be poured from the bottle and fed to the patient with a spoon.

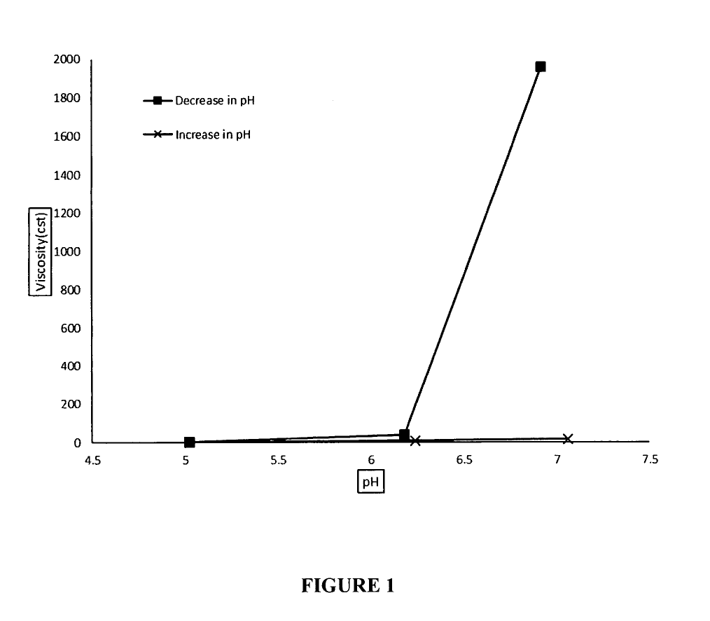

In certain embodiments, change in viscosity at various pHs was studied. The polymer was dissolved in water and alkali was added to increase the pH to 6.92 which turned the formulation to a viscous gel. Hydrochloric acid, 0.5 N, was used to decrease the pH of the formulation to 5.02 and the change in viscosity was determined. Alkali was added again to increase the pH and viscosity was determined after intervals to confirm an expected change in viscosity. FIG. 1 shows the changes in the viscosity values of the formulation with respect to the pH change.

The pH of thickened water formulation initially was around pH 7 and it was highly viscous. Based on the data produced, the viscosity in the stomach can be easily predicted. When the thickened water would enter the stomach, the acidic environment will cause a sudden decrease in viscosity. It will prevent the feeling of sateity. After a short residence time in the stomach, the formulation will pass to the deodenum, where the pH is more than the pH in the stomach. From the profile in FIG. 1, one can predict that inspite of further increase in the pH, the viscosity of the formulation would remains low. This will ensure that the formulation would not turn viscous after entering the deodenum.

In certain cases, a 0.12% formulation of the carbomer in alkali water was prepared and divided into smaller batches to determine the effect of ionic strength on the viscosity of the formulation. Sodium chloride was added to these batches in decreasing amounts. A sudden drop in the viscosity values was observed with the addition of salt. The viscosity value at different salt concentrations have been listed in Table 8.

TABLE 8
Change in viscosity with respect to salt concentration.

| Salt Concentration | Viscosity | |
|---|---|---|
| (%) | (cst) | pH |
| 0 | 1171.2 | 6.9 |
| 0.01 | 29.92 | 6.8 |
| 0.1 | 3.015 | 6.2 |
| 0.5 | 1.5 | 5.8 |

The pH values were decreased slightly by addition of salt. Inspite of bringing the pH back to 7.0, the viscosity did not increase. Presence of salts in the formulation was shown to affect the viscosity.

In another embodiment, effect of tap water was studied. Use of tap water (which may contain salts of calcium, magnesium, sodium etc. as impurities) caused the polymer to precipitate as soon as it was added to the formulation hence the polymer lost its property of forming a viscous solution. On the other hand, deionized water can help in retaining the gel-forming property and provide a solution of desired viscosity.

In another aspect, effect of use of sorbitol on the viscosity was examined. A formulation containing 0.12% polymer was prepared and divided into smaller batches. Table 9 lists the effect of sorbitol concentration on the viscosity of the formulation. It is required to estimate the change in viscosity caused due to particular concentration of sorbitol before finalizing the formulation.

TABLE 9
Effect of sorbitol concentration on viscosity

| Sorbitol | Viscosity |
|---|---|
| concentration (%) | (cSt) |
| 0 | 1153.2 |
| 2.5 | 1040.4 |
| 5.0 | 988.8 |
| 7.5 | 553.2 |

Sorbitol has some effect on the viscosity of the polymer gel. In order to attain a desired viscosity at a particular concentration of sorbitol in the formulation, it is necessary to increase the percent of polymer in the formulation.

Overall, thickened water can be prepared using the anionic polymers in water which showed unique properties. The thickened water was thick during administration helping to manage the dysphagia issues. It thinned out in the stomach thereby negating side-effects of the commercially available formulations. The proposed thickened water does not become viscous again once it reaches the duodenum and small intestinal areas.

1. A composition of thickened water formulation for the management of dysphagia which is (i) suitably thick when administered to a patient; (ii) thins out when it reaches stomach; (iii) remains thin when it reaches duodenum and small intestine.

2. The composition of thickened water formulation for the management of dysphagia in claim 1 which is in a powder form.

3. The composition of thickened water formulation for the management of dysphagia in claim 1 which is in a Ready-to-Use liquid form.

4. The powder form of thickened water formulation for the management of dysphagia as in claim 2 which is further mixed with a suitable quantity of alkaline deionized water to form thickened water.

5. The powder form of thickened water formulation for the management of dysphagia in claim 2 comprising: (i) an acid functional polymer(s) and (ii) pharmaceutically acceptable excipients.

6. The powder form of thickened water formulation for the management of dysphagia in claim 2 wherein the concentration of the acid functional polymer(s) from about 5% to about 95%.

7. The powder form of thickened water formulation for the management of dysphagia in claim 2 wherein a single or multiple acid functional polymers are used.

8. The powder form of thickened water formulation for the management of dysphagia in claim 2 wherein pH of the resultant thickened water after the addition of alkaline deionized water is from about 5 to about 8.

9. The powder form of thickened water formulation for the management of dysphagia in claim 2 wherein the viscosity of the resultant thickened water after the addition of alkaline deionized water is from about 10 cps to about 2500 cps.

10. The Ready-to-use liquid form of thickened water formulation for the management of dysphagia in claim 3 comprising: (i) an add functional polymer(s); (ii) pharmaceutically acceptable excipients and (iii) deionized water.

11. The Ready-to-use liquid form of thickened water formulation for the management of dysphagia in claim 3 wherein the concentration of the acid functional polymer(s) is from 0.001% to about 0.5%.

12. The Ready-to-use liquid form of thickened water formulation for the management of dysphagia in claim 3 wherein the composition has viscosity of about 10 cps to 2500 cps.

13. The Ready-to-use liquid form of thickened water formulation for the management of dysphagia in claim 3 wherein the composition has the pH from about 5 to about pH 8.
